(19) United States
(12) Patent Application Publication
(10) Pub. No.: US 2015/0062299 A1
(43) Pub. Date: Mar. 5, 2015

(54) QUANTITATIVE 3D-ENDOSCOPY USING STEREO CMOS-CAMERA PAIRS

(71) Applicant: The Regents of the University of California, Oakland, CA (US)

(72) Inventors: Robert George Brown, Tustin, CA (US); Alexander Kamyar Jabbari, Irvine, CA (US)

(73) Assignee: The Regents of the University of California, Oakland, CA (US)

(21) Appl. No.: 14/475,211

(22) Filed: Sep. 2, 2014

Related U.S. Application Data
(60) Provisional application No. 61/872,123, filed on Aug. 30, 2013.

(51) Int. Cl. H04N 13/02; A61B 1/06; A61B 1/05; H04N 7/18; H04N 5/374

(52) U.S. Cl. CPC H04N 13/0239; H04N 7/183; H04N 7/181; H04N 5/374; A61B 1/051; A61B 1/06; H04N 2005/2255
USPC 348/45; 348/65

(57) ABSTRACT

The present invention relates to medical devices such as endoscopes that may be used to visualize biological material during a medical procedure, such as surgery. In one embodiment, the present invention provides for an endoscope comprising one or more electronic-cameras arranged to create one or more stereo picture pairs to permit quantitative 3-dimensional (3D) imaging and analysis. In another embodiment, the present invention provides a method of imaging comprising using a 3D endoscope with one or more electronic cameras to create one or more stereo picture pairs.

### CROSS-REFERENCE TO RELATED APPLICATIONS

The present application claims the benefit of priority under 35 U.S.C. §119(e) of provisional application Ser. No. 61/872,123 filed on Aug. 30, 2013, the contents of which are hereby incorporated by reference.

### FIELD OF THE INVENTION

The invention relates to the medical device field, specifically visualization and quantification of objects.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Today, optical endoscopy is a standard tool in all hospitals, but mostly 2D versions are used. Often the intestine and colon regions are of interest, as well as nasal and sinus regions in the head. Most surgeons at present work with 2D images and must learn with difficulty how to manipulate cutting and sewing tools in that 2D image, through repeated trial-and-error learning. Accurate knowledge of the shapes, sizes, colors of obstructions and growths in internal passages are of considerable interest to surgeons. The size and shape development of such objects over time is of special interest. Extracting high-precision quantitative information from an optical fiber arrangement such as in an optical fiber endoscope is possible but difficult. Extension of that technique to surround-3D, viewing all around the endoscopic probe head, appears most unlikely to be possible, yet such a capability would be of enormous value to the surgeon for viewing side-facing and rear-facing cavities off to the side of the main passage under investigation. Thus, there is a compelling need in endoscopic medical procedures for surgeons to have 3D visual information available to them.

### SUMMARY OF THE INVENTION

Various embodiments include an endoscope comprising a plurality of electronic cameras. In another embodiment, the plurality of electronic cameras are arranged to create one or more stereo picture pairs for quantitative 3-dimensional (3D) imaging. In another embodiment, the plurality of electronic cameras incorporate electronic pixelated detector arrays. In another embodiment, the plurality of electronic cameras incorporate one or more micro-CMOS cameras. In another embodiment, the endoscope further comprises computer software processing for saving and/or analysis of quantitative 3D imaging. In another embodiment, the computer software processing involves photogrammetry, SIFT, SURF and stereo-reconstruction algorithms. In another embodiment, the endoscope further comprises a diagnostic and/or therapeutic component. In another embodiment, the diagnostic and/or therapeutic component includes a photo-dynamic therapy probe, multi-spectral spectroscopy, or hyper-spectral spectroscopy. In another embodiment, the endoscope has a probe of a diameter between 50 to 80 mm. In another embodiment, the endoscope has a probe of a diameter between 20 to 50 mm. In another embodiment, the endoscope has a probe of a diameter between 10 to 20 mm. In another embodiment, the endoscope has a probe of a diameter between 3 to 5 mm. In another embodiment, the endoscope has a probe of a diameter less than 1 mm.

Other embodiments include a device comprising one or more electronic-cameras arranged to create one or more stereo picture pairs to permit quantitative 3-dimensional (3D) imaging and analysis. In another embodiment, the cameras incorporate one or more electronic pixelated detector arrays. In another embodiment, the outputs from one or more electronic pixelated detector arrays are stored and/or processed in an electronic computer. In another embodiment, the device further comprises computer software processing of saved images. In another embodiment, the computer software processing of saved images involves photogrammetry, Scale Invariant Feature Transform (SIFT), Speeded up Robust Feature (SURF), and/or stereo-reconstruction algorithms. In another embodiment, the device further comprises acquired 3D electronic and/or computed data, that is displayed on an electronic display. In another embodiment, the acquired 3D electronic and/or computed data is both raw and processed data. In another embodiment, the device further comprises multiple 3D images as static frames or dynamic frames as in video processing and/or data capture. In another embodiment, the multiple picture-pairs acquired are used to create a composite surround-3D-image, allowing up to 4π-steradians (360 degrees in all directions) of viewing. In another embodiment, the nodal planes or principal planes of the lenses of the one or more electronic cameras are placed on a spherical (non-planar) surface to simplify the stitched 3D-image reconstruction computations and minimize image distortions. In another embodiment, the 3D images are displayed using projection 2D or 3D techniques in a CAVE-type projection display. In another embodiment, the device further comprises photodynamic therapy, and/or multi- or hyper-spectral techniques and/or laser ablation techniques simultaneously. In another embodiment, the device further comprises a component for disease-identification and/or tissue ablation.

Other embodiments include a method of imaging, comprising providing an endoscope comprising a plurality of electronic cameras arranged to create one or more stereo picture pairs, and using the endoscope to provide quantitative 3-dimensional (3D) imaging and analysis of a sample. In another embodiment, the imaging is performed in conjunction with a surgical procedure. In another embodiment, the plurality of electronic cameras incorporate one or more electronic pixelated detector arrays. In another embodiment, the outputs from one or more electronic pixelated detector arrays are stored and/or processed in an electronic computer.

Various embodiments include a method of performing a medical procedure, comprising providing a quantitative 3-dimensional (3D) endoscope comprising one or more electronic-cameras arranged to create one or more stereo picture pairs, and visualizing and/or measuring a region in a patient by using the quantitative 3D endoscope. In another embodiment, the region is the intestine and/or colon. In another embodiment, the region is the nasal and/or sinus region. In another embodiment, the quantitative 3D endoscope is used in conjunction with performing a surgical procedure. In another embodiment, data from the quantitative 3D endoscope is overlayed on 2-dimensional (2D) data. In another embodiment, the method further comprises multiple 3D views to create 3D video.

Other embodiments include a method of diagnosing a subject, comprising visualizing and/or analyzing a sample from the subject by an endoscope, wherein the endoscope comprises a plurality of electronic cameras arranged to create one or more stereo picture pairs for quantitative 3-dimensional (3D) imaging, and diagnosing the subject. In another embodiment, the endoscope has a probe of a diameter between 3 to 5 mm. In another embodiment, the endoscope has a probe of a diameter less than 1 mm. In another embodiment, the endoscope further comprises a connection to computer software processing for saving and/or analysis of quantitative 3D imaging.

Other features and advantages of the invention will become apparent from the following detailed description, taken in conjunction with the accompanying drawings, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts, in accordance with embodiments herein, geometric representation of a two-camera system imaging at two specific locations, A and C. Ranges A and C are computed by parallax differences.

FIG. 2 depicts, in accordance with an embodiment herein, 3D endoscopy diagrams, schematically at the top and geometrically below.

FIG. 3 depicts, in accordance with an embodiment herein, prototype endoscope, with two cameras encapsulated in aluminum cylinder of 25 mm diameter.

FIG. 4 depicts, in accordance with an embodiment herein, a richly-featured pistachio-nut (simulating a polyp) placed in a tube at a range of ˜60 mm for 3D-surface topography estimation.

FIG. 5 depicts, in accordance with an embodiment herein, results of imaging for the pistachio-nut (simulating a polyp). FIG. 5(a) depicts original data plot of the reconstructed surface of the pistachio-nut shown in FIG. 4. Values in mm. FIG. 5(b) depicts colored guide-lines overlaid on FIG. 5a, to aid the reader in identifying the features similarly-colored in FIG. 5c. Values in mm. FIG. 5(c) depicts colored guide-lines overlaid on FIG. 4, to aid the reader in identifying the features similarly-colored in FIG. 5b.

FIG. 6 depicts, in accordance with an embodiment herein, surround-3D camera-geometries for an optical endoscope.

FIG. 7 depicts, in accordance with an embodiment herein, the stereo image-processing software flowchart.

FIG. 8 depicts, in accordance with an embodiment herein, capture GUI. The user labels the two video feeds above each preview image. Option on right hand side are for “Begin Capture”, “Stop Capture”, “Save Images”, “Watch Video”.

FIG. 9 depicts, in accordance with an embodiment herein, calculation GUI. On the left hand side the user selects the two image pairs from the drop boxes. On the right hand side “SIFT” goes through and finds all matched points. “Calculate” will find all 3-D co-ordinates to the matched points and display on graph.

FIG. 10 depicts, in accordance with an embodiment herein, SIFT results. The turquoise lines are created to show graphically the connection from a match in one picture to its corresponding match in the other picture.

FIG. 11 depicts, in accordance with an embodiment herein, 3-D stem-plot of the computed x,y,z results.

FIG. 12 depicts, in accordance with an embodiment herein, surface plot of FIG. 11. The software takes all of the selected points and uses built-in MATLAB functions to determine a surface-plot over the stemmed-data.

FIG. 13 depicts, in accordance with an embodiment herein, the stem-plot and surface-plot overlaid on top of one another.

FIG. 14 depicts, in accordance with an embodiment herein, results of SURF before removing false positive matches.

FIG. 15 depicts, in accordance with an embodiment herein, SURF results after removing false positive matches.

FIG. 16 depicts, in accordance with an embodiment herein, SURF results in stem form.

FIG. 17 depicts, in accordance with an embodiment herein, surface plot of SURF data.

FIG. 18 depicts, in accordance with an embodiment herein, surface plot of Extrapolated data every 7 pixels (ie, under-sampled). Values in mm.

FIG. 19 depicts, in accordance with an embodiment herein, correctly sampled, as used in FIG. 5 herein. Values in mm.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described.

As disclosed herein, the inventors developed a novel and alternative approach to 3D endoscopy. In accordance with various embodiments herein, the inventors use multiple micro-CMOS cameras, acquisition of stereo-picture pairs—and computer software processing of those stereo-picture pairs, for example through photogrammetry, to extract quantitative 3D information. In one embodiment, the 3D-endoscope is based on the use of micro-CMOS detector array technologies arranged in an instrument of the size of a regular endoscope used for intestinal observations and surgery.

As further disclosed herein, the inventors solve the problems of giving the surgeon quantitative 3D-vision inside cavities in the human body, so that precision assessment of obstructions and growths for example, can be made, and surgery may be conducted in 3D-instead of the more common 2D which is particularly difficult for surgeons. In one embodiment, following a one-time calibration, semi-automated quantitative plots of the 3-D landscape some 50 mm to 60-mm range ahead of the endoscope can be viewed on a 3D screen (for example, on a laptop computer) overlaid on and/or side-by side with the scene being observed.

In one embodiment, range estimates are of order 150-microns accuracy using VGA CMOS cameras. In another embodiment, the range estimates may be improved to 45-micron minimum-error—when HDTV-format cameras are used for example, because of the smaller pixels available. These accuracies are sufficient for surgeons to have excellent 3D knowledge of obstructions or growths that they may be dealing with, and for tracking changes over time through repeated measurements and comparisons. In one embodiment, medical endoscopy is enhanced through 3D vision and spectroscopy, in-situ diagnostics of growths, etc. In another embodiment, there is a reduction in costs of 3D-vision as the well as enhanced ruggedness and portability by elimination of optical fiber endoscope aspects. In another embodiment, the invention may be further extended to 3D-panoramic and surround 3D through the use of multiple-cameras embedded in the endoscopic probe. In another embodiment, the invention also has photo-dynamic therapy, laser-spectroscopic and/or laser-ablative techniques to enhance the analytical endoscopy tool-kit further.

In one embodiment, the present invention provides an endoscope made up of one or more electronic-cameras arranged to create one or more stereo picture pairs to permit quantitative 3-dimensional (3D) imaging and analysis. In another embodiment, the cameras incorporate CMOS and/or other electronic pixelated detector arrays. In another embodiment, the endoscope contains outputs from the multiplicity of detector arrays that are stored and processed in one or more electronic computers. In another embodiment, the endoscope contains computer software processing of saved images. In another embodiment, the computer software processing of saved images involves photogrammetry, SIFT, SURF and stereo-reconstruction algorithms. In another embodiment, the endoscope further includes acquired 3D electronic and computed data, both raw and processed data, that is displayed on an electronic display. In another embodiment, the endoscope further includes multiple 3D images as static frames or dynamic frames as in video processing and/or data capture. In another embodiment, the multiple picture-pairs acquired are used to create a composite surround-3D-image, allowing up to 4π-steradians (360 degrees in all directions) of viewing. In another embodiment, the endoscope includes nodal planes or principal planes of the multiple lenses that are placed on a spherical (non-planar) surface to simplify the stitched 3D-image reconstruction computations and minimize image distortions. In another embodiment, the multiple 3D images are displayed using projection 2D or 3D techniques in a CAVE-type projection display. In another embodiment, the endoscope further comprises photodynamic therapy, and/or multi- or hyper-spectral techniques and/or laser ablation techniques simultaneously in the same endoscopic probe assembly.

In one embodiment, the present invention provides a method of imaging, where an endoscope made up of one or more electronic-cameras arranged to create one or more stereo picture pairs to permit quantitative 3-dimensional (3D) imaging, is used to provide 3D imaging and analysis of a patient. In another embodiment, the imaging and analysis is performed in conjunction with a surgical procedure.

In another embodiment, the present invention provides a method of performing a medical procedure, comprising providing a quantitative 3-dimensional (3D) device comprising one or more electronic-cameras arranged to create one or more stereo picture pairs, and visualizing and/or measuring a region in a patient by using the quantitative 3D device. In another embodiment, the device is an endoscope. In another embodiment, the present invention provides a method of performing a medical procedure, comprising providing a quantitative 3-dimensional (3D) endoscope comprising one or more electronic-cameras arranged to create one or more stereo picture pairs, and visualizing and/or measuring a region in a patient by using the quantitative 3D endoscope. In another embodiment, the region is the intestine and/or colon. In another embodiment, the region is the nasal and/or sinus region. In another embodiment, the quantitative 3D endoscope is used in conjunction with performing a surgical procedure. In another embodiment, data from the quantitative 3D endoscope is overlayed on 2-dimensional (2D) data.

In one embodiment, the device or endoscope has a probe section. In one embodiment, the device or endoscope has a probe of a diameter between 80 to 100 mm. In another embodiment, the device or endoscope has a probe of a diameter between 50 to 80 mm. In another embodiment, the device or endoscope has a probe of a diameter between 20 to 50 mm. In another embodiment, the device or endoscope has a probe of a diameter between 10 to 20 mm. In another embodiment, the device or endoscope has a probe of a diameter between 5 to 10 mm. In another embodiment, the endoscope or endoscope has a probe of a diameter between 3 to 5 mm. In another embodiment, the device or endoscope has a probe of a diameter between 1 to 3 mm. In another embodiment, the device or endoscope has a probe of less than 1 mm.

As readily apparent to one of skill in the art, the device and endoscope and the endoscope probe may be varied in any number of different sizes and diameters so as to allow specific and/or customized applications for the endoscope and related devices. For example, in one embodiment, the endoscope may include a probe about 3 mm in diameter so that the endoscope may be customized for sinus applications.

As further described herein and in accordance with an embodiment herein, the inventors used two off-the-shelf CMOS cameras with a standard USB 2.0 outputs, wherein the CMOS detector was ⅙ inches in diagonal with a VGA pixel-count of 640 by 480. However, as readily apparent to one of skill in the art, any number of sized and types of electronic cameras may be used and the invention is in no way limited to only CMOS cameras, or to the sizes of CMOS cameras that are currently commercially available.

In one embodiment, the present invention provides a method of diagnosing a subject comprising obtaining a sample from the subject, and diagnosing the subject by analyzing the sample by using a device comprising one or more electronic-cameras arranged to create one or more stereo picture pairs to permit quantitative 3-dimensional (3D) imaging and analysis. In another embodiment, the device further comprises a computer software processing for saving and/or analysis of quantitative 3D imaging.

In another embodiment, the present invention provides a method of diagnosing a disease subtype in a subject comprising obtaining a sample from the subject, and diagnosing the subtype based on the analysis of the sample from the use of a device comprising one or more electronic-cameras arranged to create one or more stereo picture pairs to permit quantitative 3-dimensional (3D) imaging and analysis.

In one embodiment, the present invention provides a method of prognosing a disease in a subject comprising obtaining a sample from the subject, and prognosing a severe case of the disease based on analysis of the sample by using a device comprising one or more electronic-cameras arranged to create one or more stereo picture pairs to permit quantitative 3-dimensional (3D) imaging and analysis. In another embodiment, the device further comprises a computer software processing for saving and/or analysis of quantitative 3D imaging.

In another embodiment, the present invention provides a method of treating a subject, comprising providing a device comprising one or more electronic-cameras arranged to create one or more stereo picture pairs to permit quantitative 3-dimensional (3D) imaging and analysis, using the device for imaging and analysis of samples from the subject, and treating the subject. In another embodiment, the device further comprises a therapeutic component of the device such as a tissue ablation component.

The present invention is also directed to a kit to treat, visualize and/or analyze biological samples. The kit is useful for practicing the inventive method of diagnosing, and treating, a condition or disease. The kit is an assemblage of materials or components, including at least one of the inventive compositions.

The exact nature of the components configured in the inventive kit depends on its intended purpose. For example, in one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to diagnose a disease, or treat a tumor for example. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Motivation

Given the importance of endoscopes in surgical procedures, the inventors wanted to develop such an approach to maximize the information the inventors can acquire whilst minimizing the cost and complexity of the system. In order to minimize system and operational complexity the inventors sought a semi-automated analytical technique that allows the surgeon to obtain and read 3D data in near real time. In accordance with one embodiment, the approach herein allows one to save and process 3D-image data. This will allow the surgeon to view changes in size, shape, color, texture, etc over a desired region and over a chosen time interval. In another embodiment, other techniques may be combined into the endoscope system such as photo-dynamic therapy (PDT), or laser spectroscopy and ablation. In another embodiment, one may deploy multiple 3D views from an endoscope that might be stitched together to create surround-3D of the interior chamber being examined. This may be implemented with a walk-in 3D-CAVE so the surgeon can be surrounded by a composite 3-D image-structure.

### Example 2

### Theory

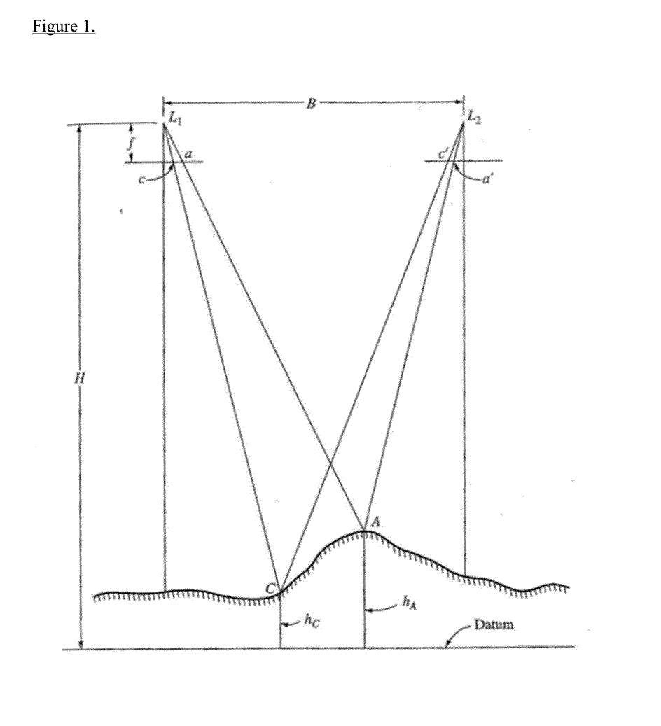

To create the quantitiative aspects of a new 3D endoscope, the inventors combined various mathematical and 3D-computing algorithms. Firstly, the inventors established photogrammetry, through which the inventors could calculate range-information at arbitrary points in the scene, selectable by the surgeon. This requires a one-time calibrated optical geometry, to be sure of the errors in estimations made later. The basic photogrammetric diagram is shown herein in FIG. 1.

The mathematics used to compute ranges A and C, and thus range-differences, is as follows, where p means parallax-distance:

$${p}_{c} == \frac{fB}{H - {h}_{C}}$$

$${p}_{a} = \frac{fB}{H - {h}_{A}}$$

$${p}_{a} - {p}_{c} = \frac{fB ( {h}_{A} - {h}_{C} )}{( H - {h}_{A} ) ( H - {h}_{C} )}$$

$${h}_{A} = {h}_{C} + \frac{Δ p ( H - {h}_{C} )}{{p}_{a}}$$

Using the relationship between the distances of the two detectors from one another, B, the distance from detectors to surface as well as focal length, the inventors are able to determine the range of what is being imaged. If h_{C} is known or made zero with respect to some datum plane established by a one-time system calibration, then relative heights h_{A} are easily calculated upon demand for any specified image points.

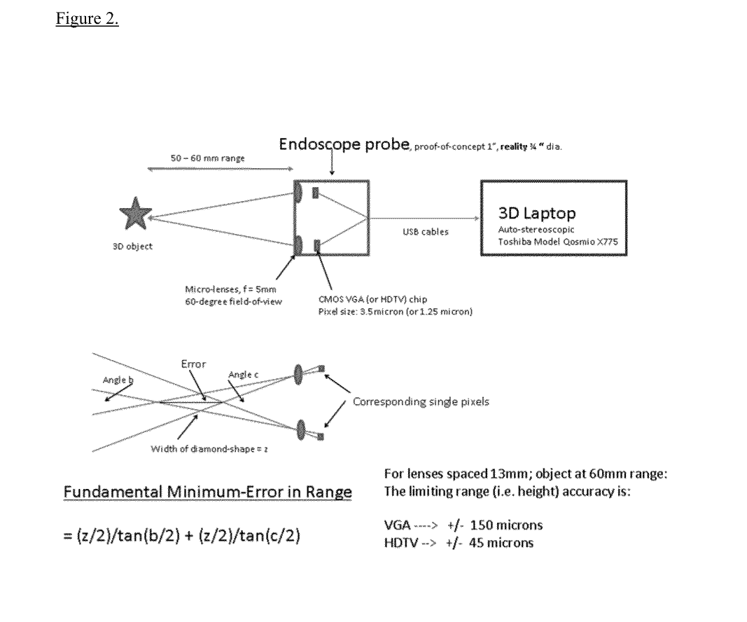

FIG. 2 shows both a schematic layout of our endoscopic probe and the geometry by which the inventors may compute its fundamental minimum error-range.

As will described in greater detail herein, the endoscope probe comprises two CMOS chips looking via 5 mm focal-length lenses at the 3D object of interest. For a certain point on the object, there will be corresponding pixels in the two CMOS pixel-arrays, whose resolution in the imaging of that object is determined by the pixels' dimensions. The inventors find that the axial or range error because of pixel dimensions is:

(z/2)/tan(b/2)+(z/2)/tan(c/2)

where z is the width of the pixel projected at the object point of interest and angles b and c are as indicated in FIG. 2. For the system operating at ˜60 mm range with a 13 mm baseline between the lenses, such minimum range-error is ˜150 microns, a little more than a human hair diameter, but in the future the inventors will reduce this to approximately 45 microns.

Other errors present themselves within this type of optical system. Of those, the most serious is radial or barrel distortion of the lenses used. This must be removed to a high degree before accurate range computations can be attempted. Two ways of removing such error are (1) direct measurement and spatial-compensation and (2) theoretical estimation based on lens parameters, with subsequent spatial correction at least to first-order.

The one-time calibration of a datum plane at range H within the optical system may be established by the use of a scale-invariant feature transform algorithm or speeded up robust feature. To calibrate, the inventors have to recognize the similar features in both images of the stereo-pair the inventors record from the two cameras spaced at a distance B. For this the inventors use two well known image processing tools Scale Invariant Feature Transform (SIFT) and Speeded up Robust Feature (SURF). SIFT is an algorithm that uses a training image to determine certain features or key points. It then looks at another image to try and match as many of the features from the training image to the second image. SIFT probabilistically employs differences in spatial-gray-scale Gaussians and least-square fitting. The version of SIFT used for the purposes here is a variation of a method by David Lowe (D. G. Lowe, ‘Object recognition from local scale-invariant features’. Proc. Int. Conf. Computer Vision. 2, pp. 1150-1157, (1999)). Challenges dealing with repetitive patterns in the images, and false-positives, may be rejected using a locality algorithm.

SURF is another feature detector in image processing and computer vision. It is based on the use of integral images and 2D Haar wavelet responses. The advantage of SURF over SIFT is faster computing time. SURF acts as a more robust faster version of SIFT. The inventors used both SIFT and SURF as they give different stereo-pair matches, which is valuable for populating the data-space prior to image processing.

To set up the SIFT and SURF calibration, first of all the inventors had to capture images with the prototype endoscope probe. First, the prototype takes two uncompressed AVI files and stores them into a folder on the computer. Then the inventors extracted all of the frames of the AVI files and store them into two new separate folders as JPGs. At this point the inventors have two folders containing sets of images from each camera. Each image has a corresponding image in the other folder (forming the pair).

Once all the main features have been matched and false positives removed, the inventors use a stereo depth reconstruction program to determine all the relevant 3-D data. This program is based on the ideas of reconstruction/triangulation using epipolar geometry (the geometry of stereo vision). Reconstruction or triangulation is done by inputting matched-pair co-ordinates from the two images and two camera matrices. The output is a set of x, y, and z values for every matched pair, x and y being the spatial coordinate in a plane parallel to the datum plane, and z the axial range at that x,y point.

With this understanding and set of techniques to hand, the inventors move to the design, implementation and measurements of our prototype 3D stereo-CMOS-camera endoscope.

### Example 3

### Experiment Design, Calibration and Computation Procedures

The inventors used two off-the-shelf CMOS cameras with a standard USB 2.0 outputs. The CMOS detector was ⅙ inches in diagonal with a VGA pixel-count of 640 by 480. Constrained by the design parameters of an endoscope, about ¾inch diameter, the inventors prototyped an aluminum-tube device of 25 mm in diameter to prove basic concepts. The two cameras are placed such that the centers of the two lenses are 13 mm apart. Both cameras are arranged to be parallel to the endoscope's central axis and are themselves parallel, as the detectors rows and columns must be parallel for both the SIFT/SURF and localized-rejection of false-positives algorithms to work effectively.

Currently the operational range of this device is 60 mm, but for the first deployable endoscope it will be reduced to be closer to the 50 mm required. The CMOS cameras did not come with detailed specifications—so in order to determine focal length, the inventors took an image of graph paper taken from a specific distance. Knowing the size of the chip the inventors are able to calculate backwards, using the lens/focal length equation, to determine the focal length of the lens. In accordance with various embodiments herein, smaller camera may be used or use same size cameras with higher pixel count of smaller pixels, to improve accuracy. In another embodiment, the endoscope diameter may be ˜3 mm diameter, for use in nasal procedures for example, and this will require use of the very smallest available CMOS sensors.

Once the two cameras are inserted into the respective slots in the aluminum tube and the cameras are connected to the computer through the USB connection, the inventors ran an image capture GUI (general user interface) that was created in MATLAB. The GUI has the user name both camera-video files before the capture sequence begins. This creates two AVI files in the parent directory and will continue capturing until the user clicks the “Stop Capture” button. After the user has finished capturing the desired video feed, all of the frames of the videos are saved into two different subfolders, one folder for each video feed. Also at this time the user is able to preview both captured video feeds in order to get a better idea of what frames might be of interest.

Next, using a second GUI, the user selects two (stereo) frames that contain a region of interest. Here the user implements the SIFT and SURF functions. In this stage SIFT and SURF examine the images and find as many matches as possible in the stereo-pair. These matches are in the form of x-y pixel-coordinates for each image. The two output variables are N×2 matrix, where N is the number of matches—and the first column represents the x-coordinate and the second column represents the y-coordinate. Because MATLAB's default is to place the origins of an image at the upper left hand pixel the inventors shift all the co-ordinates so that the center pixel is now the origin. This is done because the stereo-reconstruction program requires that the co-ordinates are relative to an origin centered in the middle of pictures.

Now the new matches (ones where the origin is at center) are sent to the stereo-reconstruction algorithm. This function returns a matrix of that is 3×N in size. Again N is the number of matches. For any given column the first row corresponds to the actual x distance from the center of the camera. This continues for the second and third rows being the y and z distances respectively. Depending on the input-order of the matches from SIFT and SURF, these distances can be relative from the image on the left side—or images on the right side. For simplicity the inventors decided to always have the results be with respect to image on the left side.

Finally the inventors made a 3-D stem graph of the data. This plots the x, y, and z distances of the results from the stereo-reconstruction. The inventors then overlay a contour-plot on top of the stem graph to show what the surface would look like.

### Example 4

### 3D Endoscope Probe Construction Details

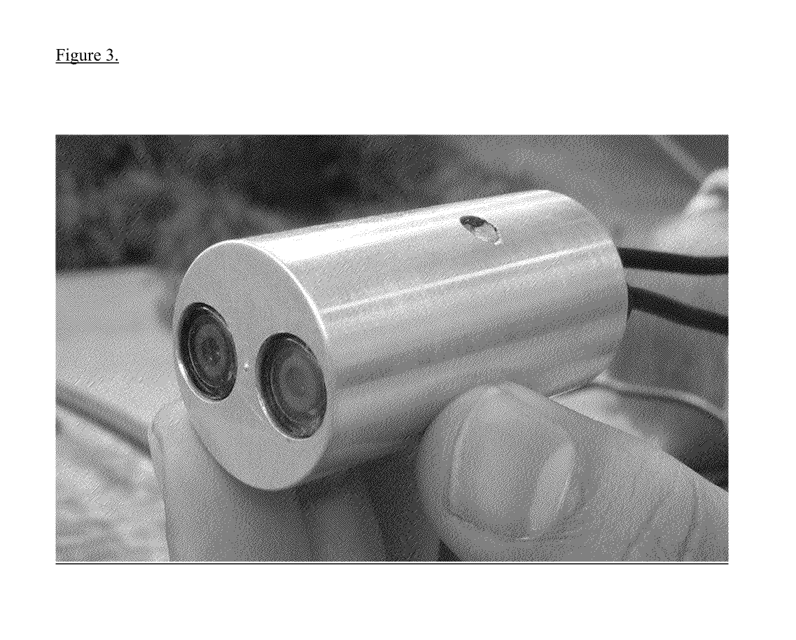

For the proof-of-concept device the inventors employed a machine-shop at UC Irvine to mill and drill two holes into an aluminum cylinder that was 25 mm in diameter. A CMOS camera in its pre-existing casing was placed into each hole and rotationally-aligned with its pair-camera to create parallel rows and columns of pixels, for reasons described earlier. Each camera comprised a CMOS sensor, a lens, 4 LED lights, as well as its output USB cable. A small threaded-hole allowed to inventors to secure the system—the hole was inserted into the side of the tube-casing so that the camera casings could be locked into place. This can be seen in FIG. 3 herein.

The USB cables exited the probe-head prototype and are fed into our laptop computer for image processing. Once images are inside the computer, the inventors used MATLAB to process the images. The three main functions that are used the inventorsre SIFT, SURF and Stereo Reconstruction. Using the built in functions in MATLAB the inventors created a surface-plot over the 3-D data to show the contours and surface of what the programs have determined in real space. This data is what surgeons can use to analyze the actual results of a procedure. All of this is done in a MATLAB GUI that was constructed using the built-in program called GUIDE.

### Example 5

### Experimental Results

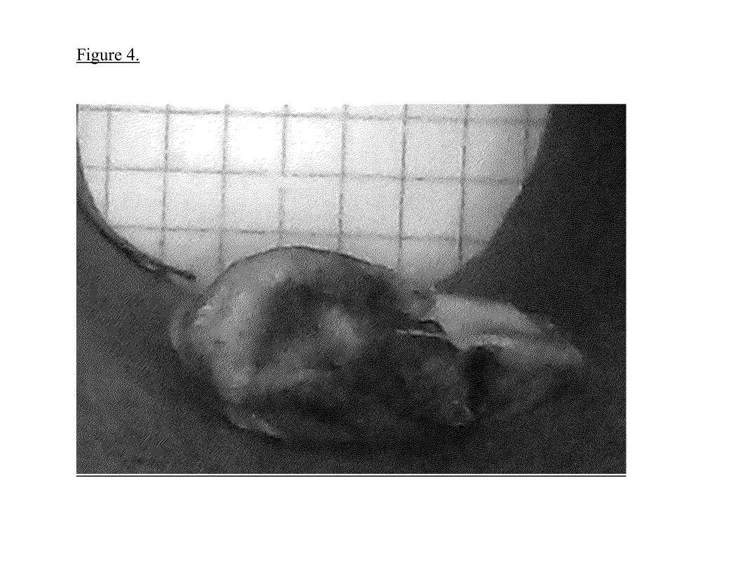

As a first experiment the inventors placed a simple biological sample (a pistachio nut) containing recognizable structure (ridges and depressions) in a 4-cm diameter tube to simulate a polyp in an intestine. The 3D-probe was placed around 60 mm from the nut and the stereo-pair photographs recorded, one of which is shown in FIG. 4 herein.

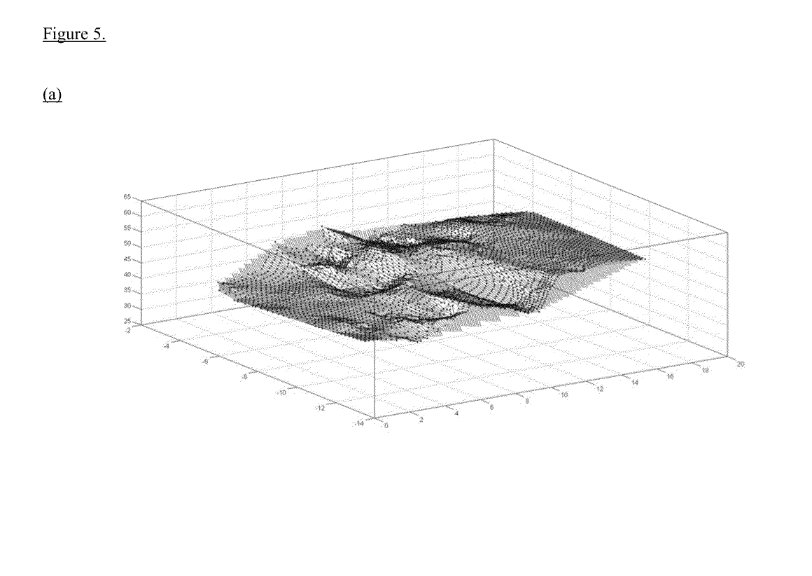

The software-processed stereo-pair, using SIFT & SURF as described previously, is shown in FIG. 5a, and also in FIG. 5b, now with colored-lines drawn onto the surface reconstruction to aid the reader in seeing the depression and ridge regions, both in that reconstruction, and next in FIG. 5c on the original picture of the nut just seen in FIG. 4. The red line outlines the ridge surrounding a heart-shaped depression and extending along the length or ‘spine’ of the nut. The orange line denotes a ridge that is perpendicular to the red-line ridge. The yellow-line traces the boundary of a second depression. All these features are clear on both the original picture of the nut and in the surface reconstruction.

FIGS. 5a & 5b show the surface-plot of extrapolated data iterated at every 23, 17, 13, 7, and 3 pixels. FIG. 5c corresponds to FIG. 4, with guidance lines.

### Example 6

### Additional Research

To date the inventors have demonstrated a working prototype for a 3-D endoscope based on dual CMOS-sensor technology. In another embodiment, the 3-D data may be overlayed on top of the original 2-D image. This will allow surgeons better to see dimensional data at the desired locations, especially object-surface range variations. In another embodiment, the user may also implement surgeon-selectable height-reports for specifically-chosen locations in the image. In another embodiment, the invention further provides displaying the 3D-reconstruction on the 3D-autostereoscopic laptop screen to aid viewing, as well as record, process and display 3D-stereo movies.

In another embodiment, the present invention may be used for and in conjunction with testing of any number of biological materials, including human biological materials. Structured-illumination techniques may also be included into the 3D probe.

In one embodiment, the whole system may be reduced down to ¾″ diameter, compatible with acceptable endoscope dimensions. Similarly, The inventors are then expecting to use smaller and higher-resolution cameras, also multiple camera views in a single endoscope probe, to permit the creation of surround-3D—for viewing sideways and behind the probe into side-cavities. Further, the inventors anticipate a surround-3D display into which the surgeon can enter to get a view from ‘inside’ the patient.

Similarly, in one embodiment, the present invention provides a device that is on the order of ˜3 mm diameter for nasal and sinus examinations—also, again to be able to create a surround 3-D environment for a doctor/surgeon to be able to walk into and be surrounded by what is in the passage being observed.

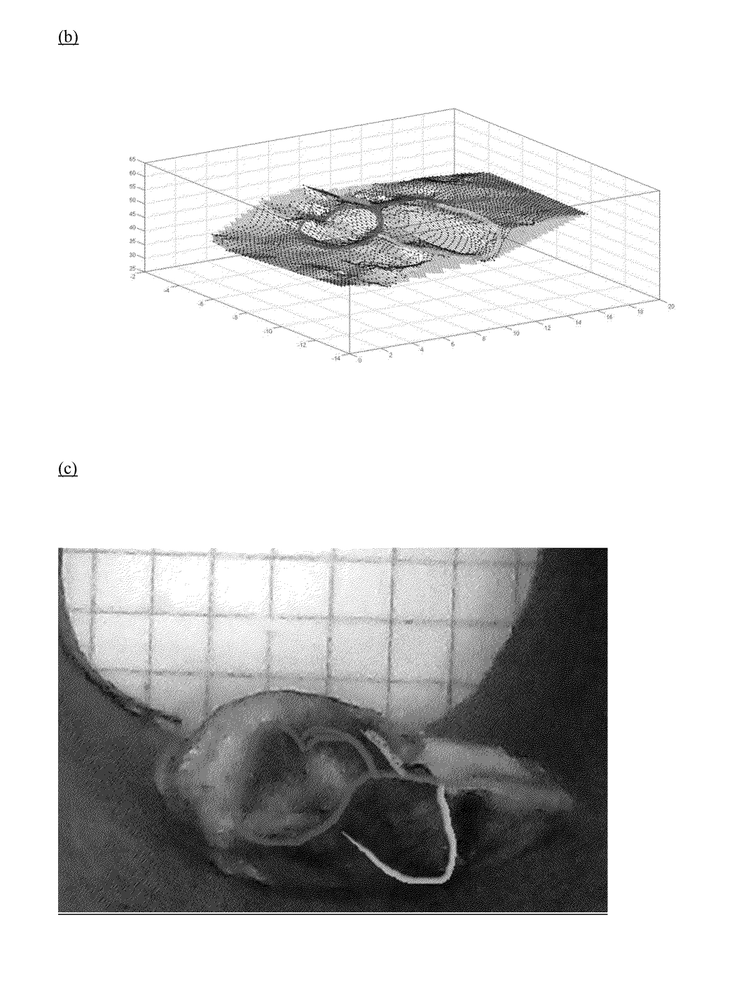

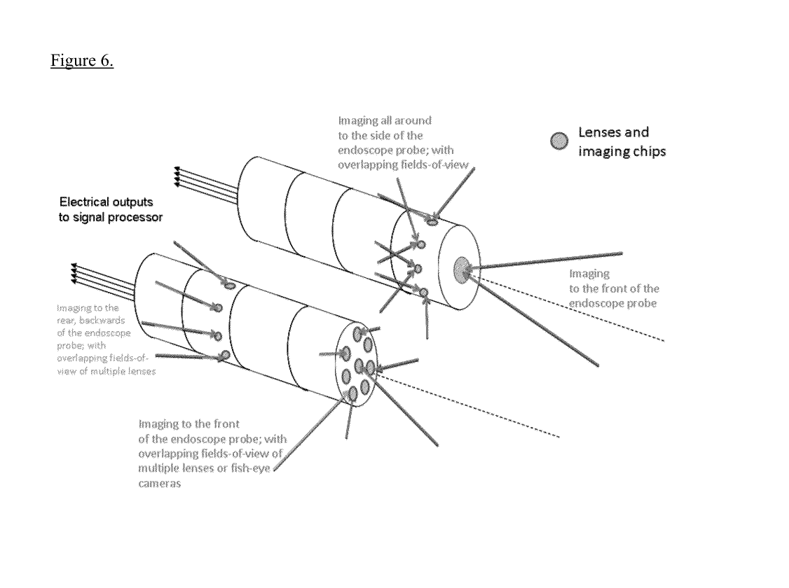

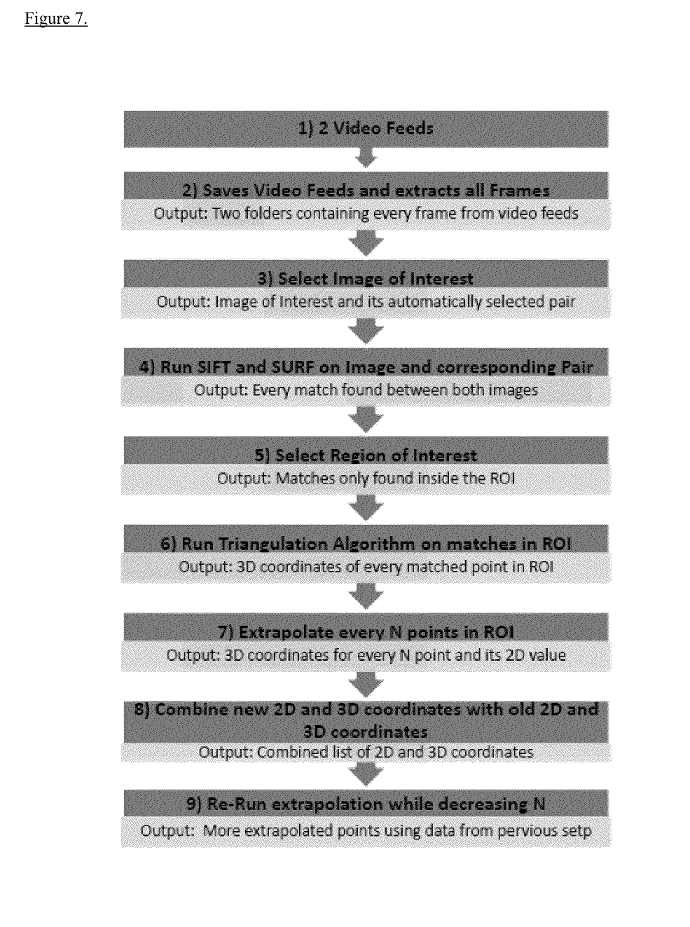

The inventors also anticipate 3-D scene-stitching—and to create stitched 3-D video. To acquire the multiple 3D views, the inventors envisage constructing more complicated probes such as those shown schematically in FIG. 6 herein.

In another embodiment, diagnostic and/or therapeutic tools may also be included into this endoscope probe, such as photo-dynamic therapy probes, spectroscopy such as multi-spectral and perhaps hyper-spectral for disease-identification—and tissue ablation.

In the left-hand probe schematic in FIG. 6 herein, the inventors see both imaging to the front of the endoscope probe and imaging to the rear, backwards of the endoscope probe, both instances using over-lapping fields-of-view of multiple lenses The arrows indicate the directions of incoming light to be imaged. Electrical outputs from each of the cameras whose images overlap are sent through the rear of the multi-view probe assembly to a computer and storage arrangement for data and image processing to recover the stitched, multiple 3D views—and then to display them.

A similar arrangement is made for the right-hand probe schematic in FIG. 6—such that imaging all around the side of the endoscope probe is achieved from side-ways looking lens/camera locations—whose fields-of-view are arranged to overlap. Imaging to the front of the probe can be through one or more lens/camera arrangements, as discussed previously. Electrical output and processing may be as further described herein.

With regard to computational-imaging involved in stitching-together multiple 3D images, in one embodiment the present invention provides for use of spherical geometry to ease the computations and reduce the distortions in the final composite 3D image. The nodal planes or principal planes of the multiple-lenses may be placed on a spherical surface.

### Example 7

### Presentation of the Results to the User: The General User Interface (GUI)

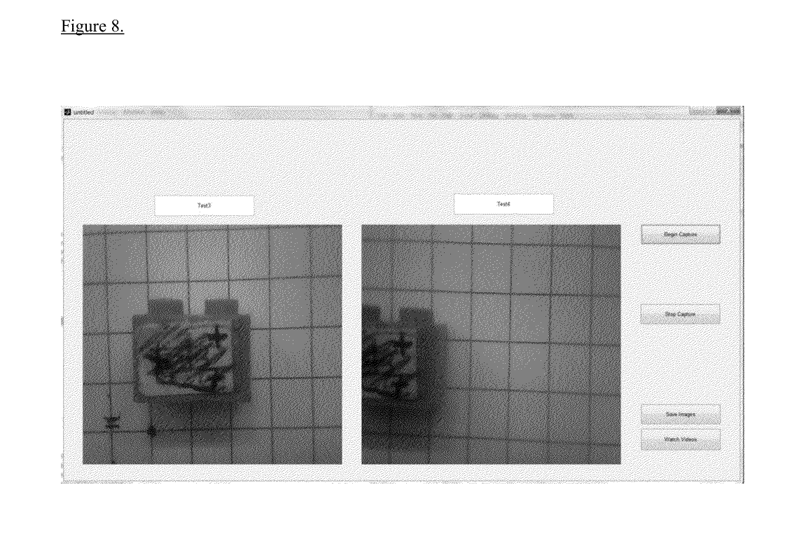

The first GUI can be seen in FIG. 8 herein. There are two windows inside the GUI that show the camera feeds from the right and left cameras, respectively. There are several user options on the right hand side. “Begin Capture” starts the camera capture mode. “Stop Capture” completes the capture sequence and saves the AVI files to the parent folder. “Save Images” creates two new subfolders in the parent directory and saves every frame into them. Finally, “Watch Video” implements the two built-in preview displays in MATLAB. Again, there is a new window for each video feed. At the bottom there is the number assigned to the frame being viewed, so the user can get a better idea of which frames have the regions of interest.

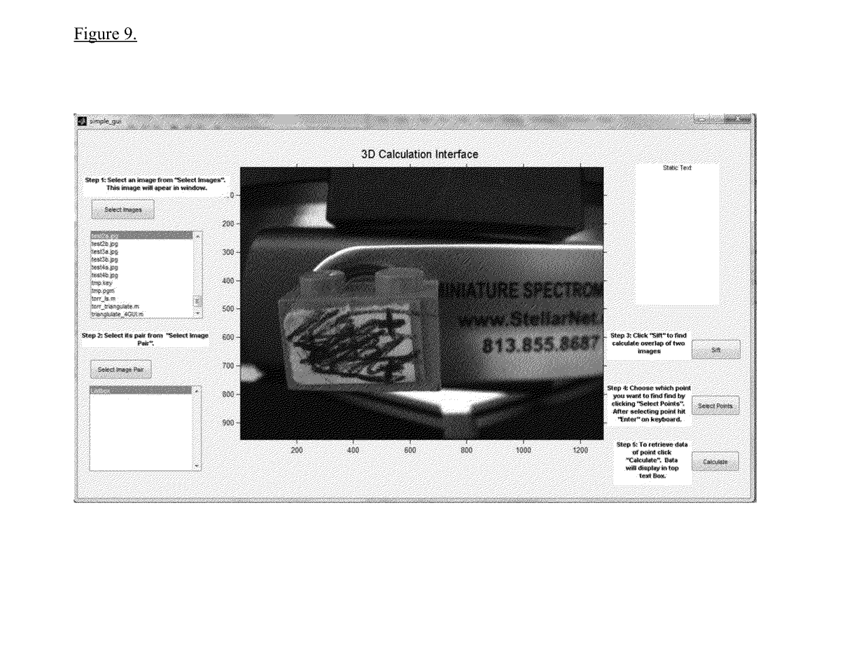

FIG. 9 herein is the second GUI the inventors created. It permits calculation of the user-chosen 3-D co-ordinates. The user follows the instructions in Steps 1-5 as shown. On the left hand side the user selects the two image pairs from drop boxes. On the right hand side, selecting “SIFT” causes the software to search and find all possible matched points using SIFT and SURF. “Calculate” will find all 3-D co-ordinates for the matched-points, and display them on a graph along with the extrapolated data.

### Example 8

### Software Algorithms and Procedures

To test the prototype endoscope, the inventors presented it with both flat and structured surfaces. The most instructive surface was a Lego block with a few lines drawn on it. The lines are included so that SIFT and SURF would have an easier time identifying and matching points on the two images. When imaging something internally in the body, there may be enough different features that this step would not be needed. If there are insufficient features, then SIFT and SURF are useful only for datum-plane calibration, and user-selected image-point matches become necessary for the extraction of range-information at desired locations of interest.

### Example 9

### SIFT

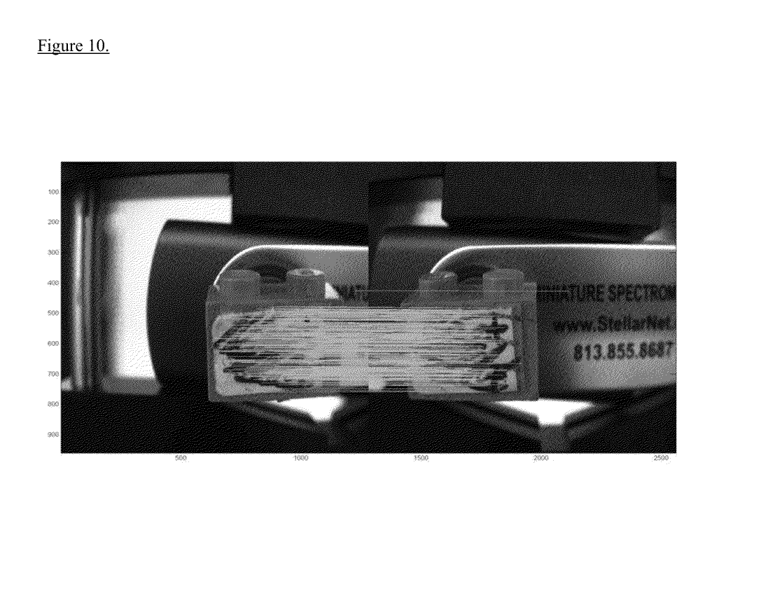

The output of SIFT is shown in FIG. 10 herein. In this image the left and right camera images are shown butted-together. The horizontal turquoise-blue lines are a generated representation showing a point in the left-hand image and its matched (corresponding) point in the right-hand image.

FIG. 11 represents the 3-D co-ordinates of every matched point, with the z values displayed as vertical stems. All these measurements are with respect to camera on the left-hand side. Each stem represents an x, y value from the center of the camera. Specifically, the z value (vertical axis) is the distance of the chosen point from the camera lens-center, in millimeters.

Note carefully in FIG. 11 the far right-hand-side point that is the outlier point seen in the top-most turquoise line of FIG. 10. This is a match found not on the plane of interest, but on the support structure behind the target. It was measured using the software to be 8.49 mm behind the datum plane. Physical measurement by using a micrometer of this same distance 8.5 mm. This was an interesting cross-check of the accuracy of the system calibration. FIG. 12 herein shows this right-hand-side far-back calibration point even more clearly.

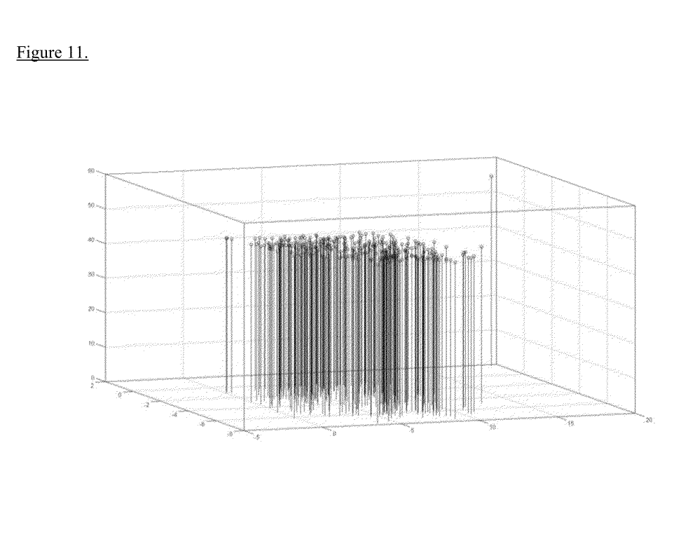

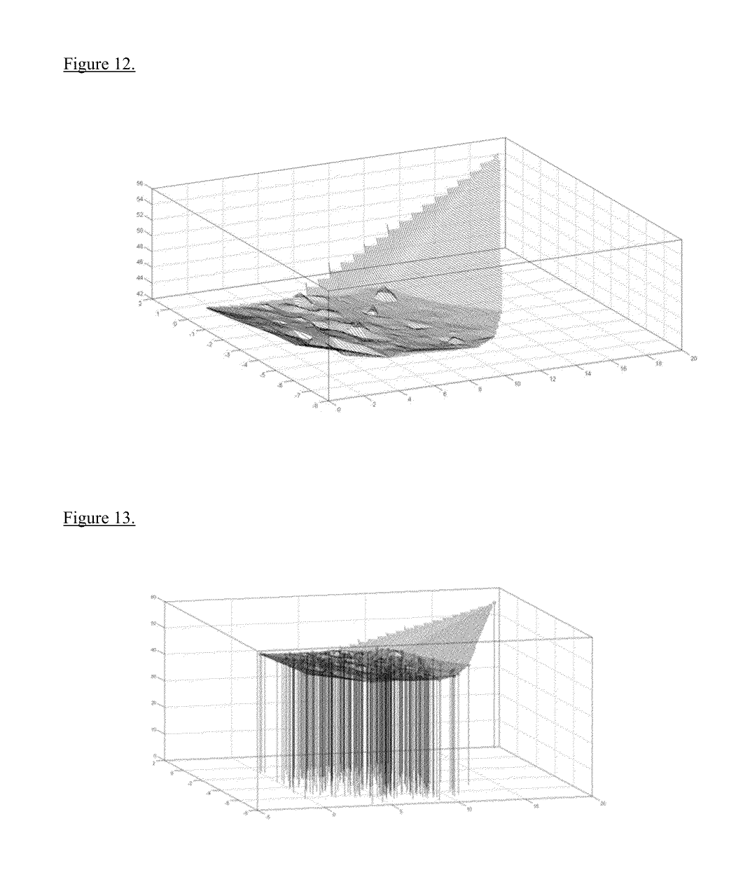

As can be seen in FIGS. 11 to 13 herein, the inventors observe both object-tilt and apparent curvature quiet clearly. Whilst the object-tilt is real, the curvature may not be real, so in future the inventors will remove barrel-distortion curvature from the original images taken by both cameras prior to any image processing.

### Example 10

### SURF

The output of SURF is shown in FIG. 14. Matlab's built in display for SURF matching overlays the two images on top of one another. In FIG. 14 the inventors observe that there is a similar outcome to SIFT matching. The display renders the matches in a similar way that SIFT does it but instead of turquoise-blue lines, Matlab uses yellow lines with green “+” and red “O” to denote matches from the left sided image and the right sided image, respectively.

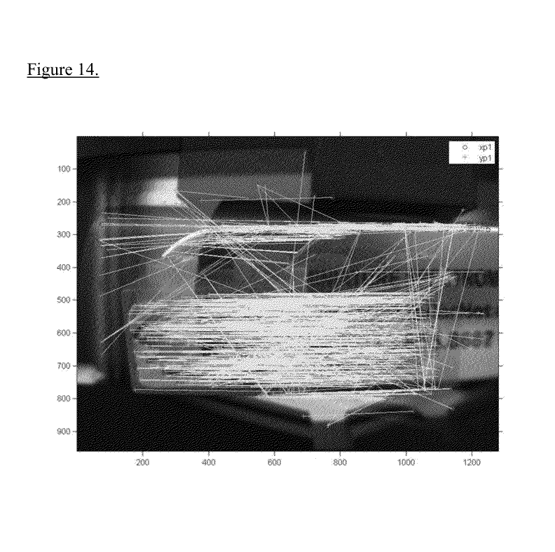

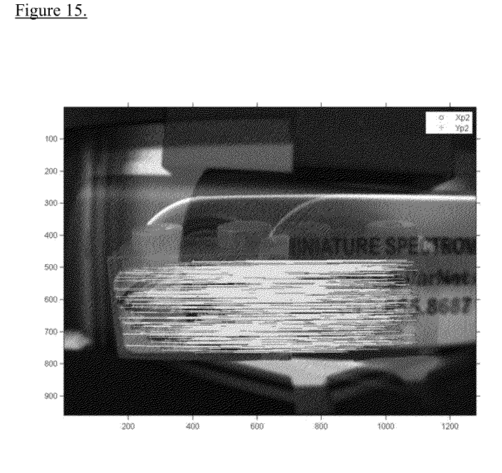

The inventors are able to use a simple filter to eliminate false matches outside of our area of interest, the Lego block. As seen in FIG. 15 herein the result from SURF post filtering, is almost the same as the SIFT data with the exception of the one outlying point on the right hand side of the FIG. 16.

The output of the SURF algorithm is similar to the output of SIFT, but contains a different set of matched points. Each match has an x-y co-ordinate in one image and a corresponding x-y co-ordinate in the image's stereo pair. Using the same triangulation algorithm, for the SURF matches, as the inventors did with the SIFT matches the inventors can obtain the 3-D co-ordinates of the SURF matches, FIG. 16. As in FIG. 11, the each stem represents an x-y co-ordinate of a match. The z dimension is the distance from the camera to the matched point in real space. Again all these measurements are relative to the image taken with the camera on the left side from FIG. 10 herein.

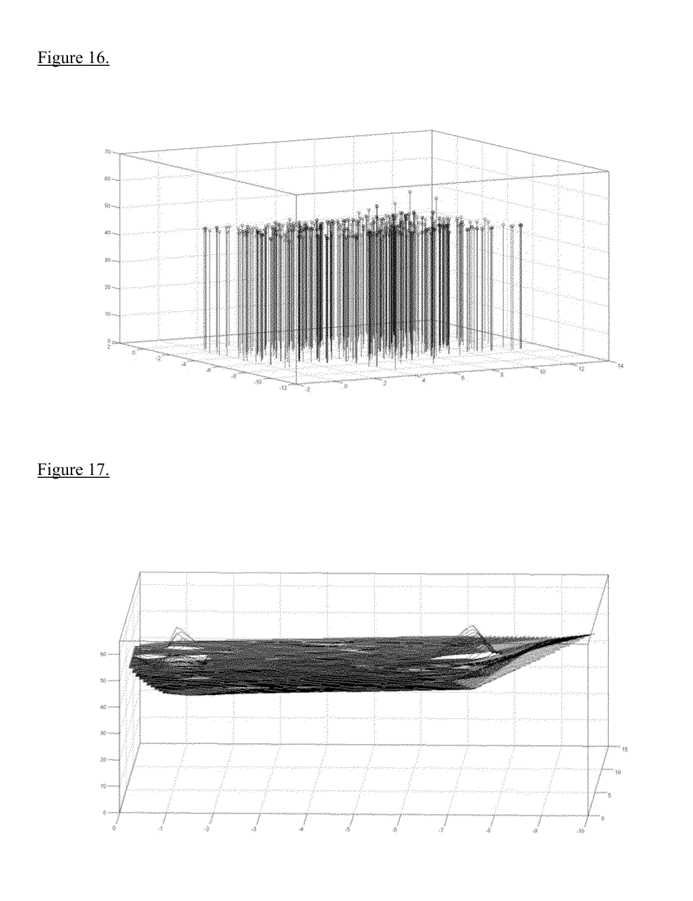

Next the inventors look at the SURF version of FIG. 12 which is shown in FIG. 17. This is the same function used to create the surface plot for the SIFT results. The inventors see that there are some peaks in the surface plot toward the left side of the graph and the right side has an upward trend for the surface. It has less of a curvature but keeps the same tilt as the SIFT surface plot displayed. Even with these inconsistencies the inventors can see that SURF data can be even just as accurate if not more so than the SIFT data.

### Example 11

### Combination of SURF AND SIFT Together with Extrapolation

After comparing the results from SIFT and SURF the inventors were reasonably assured that the two different methods would yield similar and accurate data. The next step was to place the prototype in a tube 40 mm in diameter along with an organic/natural object inside the tube. From here the inventors could incorporate both the SIFT and SURF algorithms to extract as many matches from the two images as possible. Then, using the existing data extrapolate additional points in 3-D space.

Once SIFT and SURF have run through the images the inventors compile a list of all the matched points between the two algorithms. The next step is to eliminate and double matches or matches outside of the area of interest. This is done by using the same filtering technique mentioned above. The method calls for any matches that are not inside the area of interest to be removed from the data set. Next, the new set of matches is inputted to the triangulation algorithm and the output is the x, y, and z co-ordinates, in 3D space, for all of the matched points.

To extrapolate new points the inventors use a sum of the three nearest neighbors. This says; given any point, in 2D space, a sum can be applied to the three nearest neighbors in order to generate a new point in 3D space.

To achieve this, each point in the region of interest is inputted to the nearest neighbor function. The output yields the co-ordinates, in 2-D space, of the three nearest neighbors from the existing SIFT and SURF results. Then the inventors solve the linear equation, Ax=B. The first row of A contains the x-coordinates from the nearest neighbors, the second row contains the y-coordinates of the same neighbors, and the last row is the constraint on the variables all set to 1. Matrix ‘x’ is a column vector of the three variables; Alpha, Beta, and Gamma. Finally B is another column vector of the x and y co-ordinates of the inputted point and 1 (such that the sum of the coefficients/variables sum to 1). This equation yields us a value for Alpha, Beta, and Gamma.

The inventors then scale the nearest neighbors' x, y, and z co-ordinates, in 3-D space, by Alpha, Beta, and Gamma. Meaning the nearest neighbor's x, y, and z co-ordinates are scaled by Alpha. The second nearest neighbor's components are scaled by Beta and finally the third neighbor is scaled by Gamma. The new extrapolated co-ordinates are the sum of the x-coordinates, y-coordinates, and z-coordinates In order to increase the accuracy of the extrapolated data the inventors implemented an iterative process. This allows for an incremental decrease in the number of pixels between each extrapolated point. After each iterative cycle the newly calculated 2D and 3D data is added to the existing 2D and 3D data. In each subsequent iteration there are more neighbors to choose from, decreasing the distance between a selected point and its neighbors.

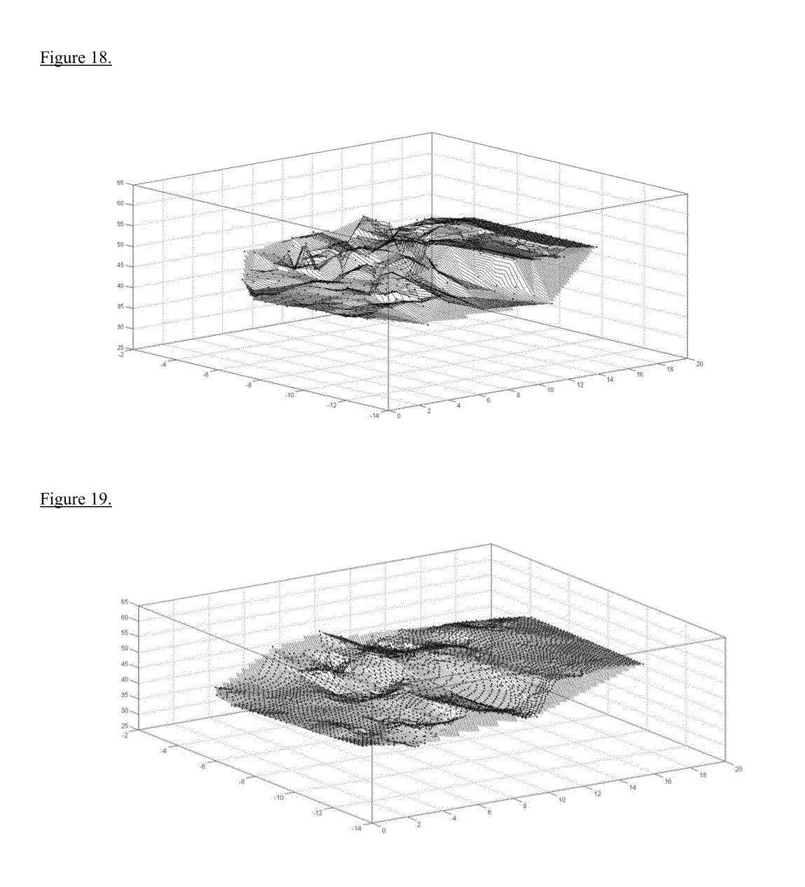

As seen in FIG. 18 herein, when the data is under-sampled at every 7 pixels, the difference in heights becomes less smooth and steeper. This causes the output to have a triangular look that does not accurately represent the surface of region of interest. The best results came when 5 iterations were done at every 23, 17, 13, 7, and 3 pixels. In FIGS. 18 and 19 herein the z-coordinates have been subtracted from 100 to show the surface as if a viewer was looking straight at the region of interest. All axis-values are in mm.

To insure that data with high error is not being added in each iteration cycle the inventors implemented two filters. The first was an area-thresholding filter. If the area between the three nearest neighbors was too small (meaning at least two points are too close together or that the points are on the same line) the data would be distorted. By setting a minimum area and finding a fourth nearest neighbor, the number of artifacts and high error points decreased. The second filter was a determinant check. If the determinant of matrix A was 0 or very close to 0 then the matrix would be linearly dependent. All data that came from a matrix with a determinant of 0 was discarded in each iteration.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s). The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. An endoscope comprising a plurality of electronic cameras.

2. The endoscope of claim 1, wherein the plurality of electronic cameras are arranged to create one or more stereo picture pairs for quantitative 3-dimensional (3D) imaging.

3. The endoscope of claim 1, wherein the plurality of electronic cameras incorporate electronic pixelated detector arrays.

4. The endoscope of claim 1, wherein the plurality of electronic cameras incorporate one or more micro-CMOS cameras.

5. The endoscope of claim 1, further comprising computer software processing for saving and/or analysis of quantitative 3D imaging.

6. The endoscope of claim 5, wherein the computer software processing involves photogrammetry, SIFT, SURF and stereo-reconstruction algorithms.

7. The endoscope of claim 1, further comprising a diagnostic and/or therapeutic component.

8. The endoscope of claim 7, wherein the diagnostic and/or therapeutic component includes a photo-dynamic therapy probe, multi-spectral spectroscopy, or hyper-spectral spectroscopy.

9. The endoscope of claim 1, wherein the endoscope has a probe of a diameter between 50 to 80 mm.

10. The endoscope of claim 1, wherein the endoscope has a probe of a diameter between 20 to 50 mm.

11. The endoscope of claim 1, wherein the endoscope has a probe of a diameter between 10 to 20 mm.

12. The endoscope of claim 1, wherein the endoscope has a probe of a diameter between 3 to 5 mm.

13. The endoscope of claim 1, wherein the endoscope has a probe of a diameter less than 1 mm.

14. A device comprising one or more electronic-cameras arranged to create one or more stereo picture pairs to permit quantitative 3-dimensional (3D) imaging and analysis.

15. The device of claim 14, wherein the cameras incorporate one or more electronic pixelated detector arrays.

16. The device of claim 15, wherein the outputs from one or more electronic pixelated detector arrays are stored and/or processed in an electronic computer.

17. The device of claim 14, further comprising computer software processing of saved images.

18. The device of claim 17, wherein the computer software processing of saved images involves photogrammetry, Scale Invariant Feature Transform (SIFT), Speeded up Robust Feature (SURF), and/or stereo-reconstruction algorithms.

19. The device of claim 14, further comprising acquired 3D electronic and/or computed data that is displayed on an electronic display.

20. The device of claim 19, wherein the acquired 3D electronic and/or computed data is both raw and processed data.

21. The device of claim 14, further comprising multiple 3D images as static frames or dynamic frames as in video processing and/or data capture.

22. The device of claim 14, wherein the multiple picture-pairs acquired are used to create a composite surround-3D-image, allowing up to 4π-steradians (360 degrees in all directions) of viewing.

23. The device of claim 14, wherein the nodal planes or principal planes of the lenses of the one or more electronic cameras are placed on a spherical (non-planar) surface to simplify the stitched 3D-image reconstruction computations and minimize image distortions.

24. The device of claim 14, wherein the 3D images are displayed using projection 2D or 3D techniques in a CAVE-type projection display.

25. The device of claim 14, further comprising photodynamic therapy, and/or multi- or hyper-spectral techniques and/or laser ablation techniques simultaneously.

26. The device of claim 14, further comprising a component for disease-identification and/or tissue ablation.

27. A method of imaging, comprising:
providing an endoscope comprising a plurality of electronic cameras arranged to create one or more stereo picture pairs; and
using the endoscope to provide quantitative 3-dimensional (3D) imaging and analysis of a sample.

28. The method of claim 27, wherein the imaging is performed in conjunction with a surgical procedure.

29. The method of claim 27, wherein the plurality of electronic cameras incorporate one or more electronic pixelated detector arrays.

30. The method of claim 29, wherein the outputs from one or more electronic pixelated detector arrays are stored and/or processed in an electronic computer.

31. A method of performing a medical procedure, comprising:
providing a quantitative 3-dimensional (3D) endoscope comprising one or more electronic-cameras arranged to create one or more stereo picture pairs; and
visualizing and/or measuring a region in a patient by using the quantitative 3D endoscope.

32. The method of claim 31, wherein the region is the intestine and/or colon.

33. The method of claim 31, wherein the region is the nasal and/or sinus region.

34. The method of claim 31, wherein the quantitative 3D endoscope is used in conjunction with performing a surgical procedure.

35. The method of claim 31, wherein data from the quantitative 3D endoscope is overlayed on 2-dimensional (2D) data.

36. The method of claim 31, further comprising multiple 3D views to create 3D video.

37. A method of diagnosing a subject, comprising:
visualizing and/or analyzing a sample from the subject by an endoscope, wherein the endoscope comprises a plurality of electronic cameras arranged to create one or more stereo picture pairs for quantitative 3-dimensional (3D) imaging; and
diagnosing the subject.

38. The method of claim 37, wherein the endoscope has a probe of a diameter between 3 to 5 mm.

39. The method of claim 37, wherein the endoscope has a probe of a diameter less than 1 mm.

40. The method of claim 37, wherein the endoscope further comprises a connection to computer software processing for saving and/or analysis of quantitative 3D imaging.
